(19) United States
(12) Patent Application Publication
(10) Pub. No.: US 2016/0106936 A1
(43) Pub. Date: Apr. 21, 2016

(54) Personal Vaporizer Having Controlled Usage

(71) Applicant: Breathe eCigs Corp., Knoxville, TN (US)

(72) Inventor: Josh Kimmel, Knoxville, TN (US)

(21) Appl. No.: 14/817,562

(22) Filed: Aug. 4, 2015

Related U.S. Application Data
(63) Continuation-in-part of application No. 14/519,130, filed on Oct. 21, 2014.

(51) Int. Cl. A61M 15/00; G05B 19/042; A61B 5/117; A24F 47/00; A61M 15/06

(52) U.S. Cl. CPC A61M 15/0001; A24F 47/008; A61M 15/06; A61B 5/1172; G05B 19/042; A61M 2205/60; A61M 2205/35; A61M 2205/8206; G05B 2219/25338

(57) ABSTRACT

A personal vaporizer including a cartridge configured to accommodate a plurality of chambers each configured to contain a liquid material, an atomizer configured to vaporize the liquid material so as to be inhaled by a user, a controller configured to control the atomizer, and one or more sensors configured to be in electrical communication with the controller, wherein the controller is configured to control the atomizer to vaporize the liquid material only when an authorization condition is satisfied, the authorization condition being determined according to signals received from the one or more sensors.

### CROSS-REFERENCE TO RELATED APPLICATIONS

This application is a Continuation-In-Part of U.S. application Ser. No. 14/519,130, filed on Oct. 21, 2014.

### FIELD OF INVENTION

The present general inventive concept relates to personal vaporizers, and, more particularly, components and methods to provide control the vaporization of liquid materials contained in personal vaporizers.

### BACKGROUND

Personal vaporizers, which are devices that typically simulate the feeling of smoking tobacco or other substances, are becoming more and more popular, as evidenced by the rapidly increasing sales of the devices over the last several years. Personal vaporizers are known by many names, such as electronic cigarettes, inhalation devices, electronic vaporizers, personal vapor inhaling units, electronic nicotine delivery systems (ENDS), etc., but typically function to convert a liquid material into vapor to be inhaled by a user. This action may be to deliver nicotine, flavored or non-flavored in its liquid form, to provide the sensation of smoking cigarettes. Or, as previously noted, the device may be used to ingest other substances.

Electronic cigarettes, invented in 2003, and maybe the most recognized form of personal vaporizer, are typically battery powered devices designed to provide inhaled doses of vaporized nicotine. Generally, the typical electronic cigarette will be made of three parts: (1) a power source, conventionally a battery or wired connection that draws power from a USB port of another electronic device; (2) an atomizer, which is the part of the electronic cigarette that contains the heating element to vaporize the liquid material; and (3) a cartridge, which is the part of electronic cigarette that contains the liquid material, sometimes called the liquid storage material or e-liquid. This liquid material typically contains an agent to produce the vapor, such as propylene glycol, or vegetable glycerin as an alternative for users with allergies or sensitivity to propylene glycol, to which nicotine and flavorings are added. “Cartomizers” are electronic cigarettes that combine the atomizer and cartridge into one piece. When fully assembled, the electronic cigarette tip will typically turn a color when a user is inhaling, to indicate that the device is functioning.

These so-called electronic cigarettes are widely considered a promising development in assisting people to quit smoking traditional tobacco cigarettes. Electronic cigarettes have some benefits over traditional cigarettes by offering, among other things: (a) nicotine delivery in a format (aroma, feel, and look) that mimics smoking and that can help smokers reduce and eliminate their addiction; (b) a socially acceptable non-medical image which enables users to retain their smoker identity but without the risks of smoke; (c) a relatively inexpensive alternative to traditional cigarettes; and (d) increasing consumer demand and popularity with younger adults, traditional smokers, and nonsmokers alike, which has, in turn, led to a more positive reshaping of the tobacco industry, with major manufacturers and startup companies investing in the advancement of the technology and the goodwill of the overall industry.

Use of personal vaporizers is a relatively recent phenomenon, and evidence to date of the health effects of personal vaporizers is scarce. Two particular concerns about these products relate to the potential health hazards of personal vaporizers, specifically the exposure to first and second-hand users of the vaporized nicotine emissions, and the elements of the vaporized material other than the nicotine. Related to these concerns, one unfortunate aspect of the conventional personal vaporizer is that the typical device can be used by any person who simply inhales normally on the mouthpiece of the assembled personal vaporizer, assuming a fully or partially charged device loaded with a liquid material, is able to inhale the vaporized substance. Thus, children or other users not meant to operate the device may be able to inhale vaporized material to which they should not be exposed. Also, while the tip of a typical electronic cigarette may turn a particular color while the device is in use, and may repeatedly flash as a warning to a user that inhales in excess (for example, more than 15 times in a 60-second period), aside from the repeated flash there is typically no practical function on the electronic cigarette that will reduce the amount of exposure a user has to vaporized nicotine emissions inhalation. Therefore, a personal vaporizer having a control feature to limit usage of the device may be desirable.

Another use of personal vaporizers may be to deliver drugs for medicinal purposes. Vaporized medicinal drugs may be ingested more pleasantly, especially when prescribed in large quantities, by inhaling them in a vapor form. Such ingestion may be even more pleasant for the user if the vaporized medicinal substance were able to be flavored in a similar manner to the nicotine inhaled by a typical personal vaporizer user. However, due to the nature of medicinal drugs, such as the mere access to, and preparation of, controlled substances, as well as possible unwanted chemical reactions of mixing a liquid medicinal drug with a flavoring or other substance, the conventional personal vaporizers and cartridges are not able to provide assistance in this manner. Therefore, a personal that would be able to ease the taking of prescription or other medicinal substances may be desirable.

### BRIEF SUMMARY

According to various example embodiments of the present general inventive concept, a personal vaporizer may be provided with one or more lockout sensors that allow the personal vaporizer to operate, and thus vaporize a liquid material, only when a predetermined authorization condition is satisfied. According to other various example embodiments, a personal vaporizer may be provided with a cartridge having two or more chambers in which different liquid materials may be respectively stored. According to still other various example embodiments, a modular multi-chamber cartridge in which two or more different liquid materials may be respectively stored is provided which mates with a separately provided personal vaporizer such that different liquid materials may be vaporized, either together or separately, without a user having to change liquid material cartridges connected to the personal vaporizer.

Additional aspects and advantages of the present general inventive concept will be set forth in part in the description which follows, and, in part, will be obvious from the description, or may be learned by practice of the present general inventive concept.

The foregoing and/or other aspects and advantages of the present general inventive concept may be achieved by a personal vaporizer including a cartridge configured to contain a liquid material, an atomizer configured to vaporize the liquid material so as to be inhaled by a user, a controller configured to control the atomizer, and one or more sensors configured to be in electrical communication with the controller, wherein the controller is configured to control the atomizer to vaporize the liquid material only when an authorization condition is satisfied, the authorization condition being determined according to signals received from the one or more sensors.

The foregoing and/or other aspects and advantages of the present general inventive concept may also be achieved by a personal vaporizer including a cartridge configured to accommodate a plurality of chambers each configured to contain a liquid material, an atomizer configured to vaporize one or more liquid materials received from one or more of the chambers, a plurality of valves respectively provided to the plurality of chambers and configured to selectively control flow of the one or more liquid materials to the atomizer, and a controller configured to control the valves to allow the flow of the one or more liquid materials, and to control the atomizer to vaporize the one or more liquid materials received from the one or more of the chambers.

The foregoing and/or other aspects and advantages of the present general inventive concept may also be achieved by a cartridge to supply a liquid material to a personal vaporizer, the cartridge including a plurality of chambers each configured to contain a liquid material, a housing to at least partially enclose the plurality of chambers, and a mating portion to couple the cartridge to the personal vaporizer such that the cartridge is readily attachable and detachable to the personal vaporizer as a modular component.

Other features and aspects of the present general inventive concept may be apparent from the following detailed description, the drawings, and the claims.

### BRIEF DESCRIPTION OF THE FIGURES

The following example embodiments are representative of example techniques and structures designed to carry out the objects of the present general inventive concept, but the present general inventive concept is not limited to these example embodiments. In the accompanying drawings and illustrations, the sizes and relative sizes, shapes, and qualities of lines, entities, and regions may be exaggerated for clarity. A wide variety of additional embodiments will be more readily understood and appreciated through the following detailed description of the example embodiments, with reference to the accompanying drawings in which:

FIG. 1 illustrates a personal vaporizer according to an example embodiment of the present general inventive concept;

FIG. 2 illustrates an exploded view of some of the components of a personal vaporizer according to another example embodiment of the present general inventive concept;

FIG. 3 illustrates a block diagram of various components of a personal vaporizer according to yet another example embodiment of the present general inventive concept;

FIG. 4 illustrates a block diagram of various components of the valve control unit of FIG. 3 according to an example embodiment of the present general inventive concept; and

FIG. 5 illustrates a schematic diagram of the current generator of FIG. 3 according to an example embodiment of the present general inventive concept.

### DETAILED DESCRIPTION

Reference will now be made to the example embodiments of the present general inventive concept, examples of which are illustrated in the accompanying drawings and illustrations. The example embodiments are described herein in order to explain the present general inventive concept by referring to the figures.

The following detailed description is provided to assist the reader in gaining a comprehensive understanding of the structures and fabrication techniques described herein. Accordingly, various changes, modification, and equivalents of the structures and fabrication techniques described herein will be suggested to those of ordinary skill in the art. The progression of fabrication operations described are merely examples, however, and the sequence type of operations is not limited to that set forth herein and may be changed as is known in the art, with the exception of operations necessarily occurring in a certain order. Also, description of well-known functions and constructions may be simplified and/or omitted for increased clarity and conciseness.

Note that spatially relative terms, such as “up,” “down,” “right,” “left,” “beneath,” “below,” “lower,” “above,” “upper” and the like, may be used herein for ease of description to describe one element or feature's relationship to another element(s) or feature(s) as illustrated in the figures. Spatially relative terms are intended to encompass different orientations of the device in use or operation in addition to the orientation depicted in the figures. For example, if the device in the figures is turned over or rotated, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

It is understood that in the various example embodiments of the present general inventive concept described herein the term “personal vaporizer” shall include any device typically used to vaporize a liquid material for inhalation by a user. Such devices may be otherwise known by several different names, such as, for example, an electronic cigarette, inhalation device, electronic smoking device, electronic vaporizer, etc., that is used to vaporize a liquid material to be inhaled by a user. Likewise, although the term “vapor” is used to describe the substance and state thereof inhaled by the user of the personal vaporizer, it will be understood by one skilled in the art that the atomized and inhaled substance may otherwise be called an aerosol, mist, and so on. The personal vaporizer may also be simply called a drug delivery device. Similarly, throughout these descriptions the terms “substance”, “material”, “liquid material”, and so on may be used interchangeably to refer to the liquid contents provided to a personal vaporizer to be vaporized and inhaled by a user.

An aspect of example embodiments of the present general inventive concept may be to provide a safety lockout feature to assist and enable personal vaporizers from causing any unintended and unfortunate misuse by children or other persons that should not be using such a device, and/or to reduce or otherwise lessen the amount of unintended passive exposure to so-called “second-hand” vapor. In one example embodiment of the present general inventive concept, a personal vaporizer is equipped with a safety lockout feature in the form of two pressure sensors that must be engaged at the same time to engage the ability of the personal vaporizer to vaporize a liquid material to be inhaled by the user. In some example embodiments, both of the pressure sensors must be engaged for a predetermined amount of time to enable the vaporizing function of the device. Various other example embodiments may be provided with different and/or additional types of sensors, as described herein. In various example embodiments of the present general inventive concept, such a sensor or sensors may be provided in addition to a power switch that allows the personal vaporizer to be switched between an on and off state. Various example embodiments of the present general inventive concept may provide a personal vaporizer that switches off, cease functioning, vibrate, or the like after a predetermined amount of time, or a time mandated by law, and so on.

An aspect of example embodiments of the present general inventive concept may be to provide a personal vaporizer that stores two or more liquid materials for vaporizing. For example, the personal vaporizer may include two separate compartments, and may use one compartment for nicotine and the other compartment for a medical drug such as prescription or non-prescription medication. In various example embodiments of the present general inventive concept, the personal vaporizer may include at least one controller, such as a microprocessor, that controls the personal vaporizer such that each of the liquid materials is respectively vaporized and dispensed at a predetermined rate, or quantitative amount. For example, five micrograms of each substance may be vaporized in any particular time period, or ten micrograms of a first substance and 20 micrograms of a second substance, and so on. Various example embodiments may dispense any of a number of desired or legally mandated rates or quantitative amounts.

According to various examples of the present general inventive concept, a personal vaporizer may be equipped with a controller to control an atomizer, and one or more sensors configured to be in electrical communication with the controller, wherein the controller may be configured to control the atomizer to vaporize the liquid material only when an authorization condition is satisfied, the authorization condition being determined according to signals received from the one or more sensors. In other words, a certain condition, the authorization condition, must be met before the atomizer is able to vaporize the liquid material contained in the personal vaporizer for inhalation by a user. Such a device may prevent a child or other such unauthorized user from illicitly or otherwise inhaling the vaporized substance.

According to various examples of the present general inventive concept, a personal vaporizer may be equipped with a cartridge configured to accommodate a plurality of chambers each configured to contain a liquid material. The personal vaporizer may have a controller configured to control a plurality of respective valves to allow the flow of the one or more liquid materials, and to control the atomizer to vaporize the one or more liquid materials received from the one or more of the chambers. The liquid materials contained in the cartridge may be one or more of several different substances such as, for example, medical grade water, distilled water, non-chlorinated water, chlorinated water, vegetable glycerin, propoplyn glycol, or any other compound or non-compound liquid, as well as natural, non-natural, organic, etc., materials and various additives thereto. The materials may be medications, or have medical uses, and/or may be flavorings combined with other liquid materials.

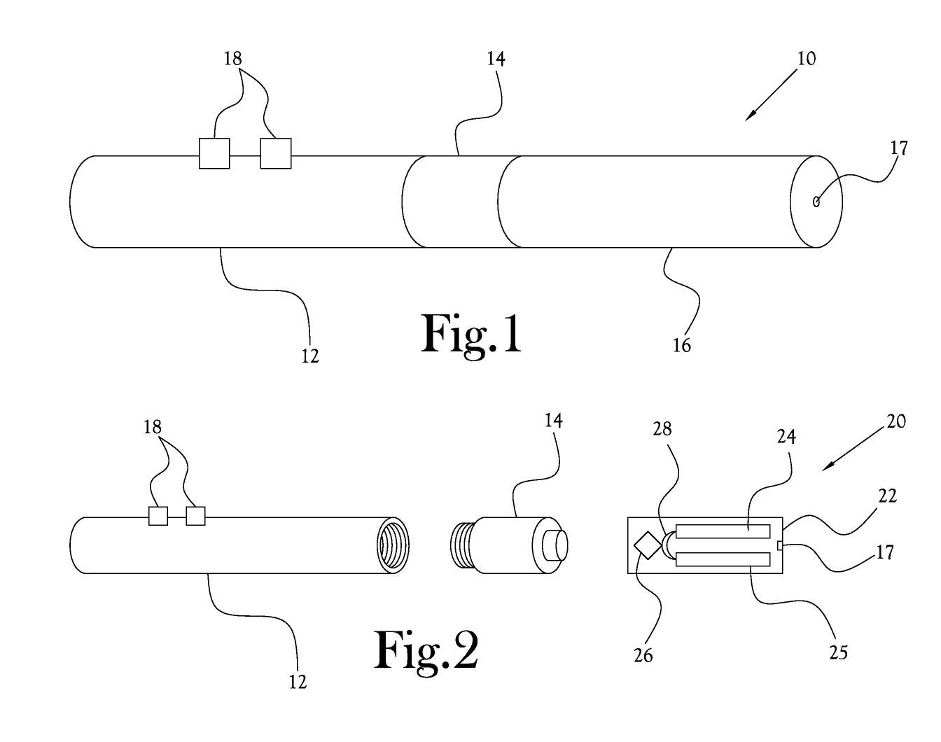

FIG. 1 illustrates a personal vaporizer according to an example embodiment of the present general inventive concept. As illustrated in FIG. 1, the personal vaporizer 10 may have a power source 12 to provide power to various components of the personal vaporizer 10, an atomizer 14 to vaporize a liquid material for inhalation by a user, and a cartridge 16 in which the liquid material may be stored in its liquid state. The cartridge 16 may be provided with an outlet hole 17 through which the user may inhale the vapor produced by the atomizer 14. A controller (e.g., a processor chip or the like) may be provided inside the personal vaporizer 10 to control the various operations of the device, such as the atomizer 14, visual indicators that indicate vaporization taking place, etc. The controller is not illustrated in FIG. 1, but it will be understood by one skilled in the art that any of a host of typical processing chips or other such processing devices may be employed as a simple controller. Similarly, particular internal characteristics of the atomizer are not illustrated in FIG. 1, as any number of conventional atomizer configurations may be employed in example embodiments of the present general inventive concept. The personal vaporizer 10 may be further provided with a power switch to switch the device between on and off states, wherein power from the power source 12 is provided to the controller and other components of the device when the personal vaporizer 10 is in an on state. According to various example embodiments, the power source 12 may be one or more batteries stored inside the personal vaporizer 10, a wired connection to, for example, a USB port or other electrical interface, a rechargeable battery module, and the like.

A pair of touch sensors 18 may be provided to the personal vaporizer 10 so as to be in electrical communication with the controller provided in the personal vaporizer 10. As illustrated in FIG. 1, the sensors 18 may be separated from one another such that the touching of one of the sensors does not inadvertently cause the touching of the other of the sensors by the same finger. In various example embodiments, both of the sensors 18 must be engaged by the user simultaneously in order for the controller to control the atomizer 14 to vaporize the liquid material in the personal vaporizer 10 to produce vapor to be inhaled by the user. Thus, the requirement that the sensors 18 be engaged functions as a safety lockout feature to prevent unwanted users, who may be unfamiliar with the safety requirement, from accessing the vaporized substance. In various example embodiments, the simultaneous touching of the sensors 18 may need to be performed in addition to an on/off switch of the personal vaporizer 10 being switched to the on position. In other various example embodiments, the simultaneous touching of the sensors 18 may operate as a substitute for an on/off switch. In various example embodiments, a predetermined series of touches to the sensors 18 may need to be performed before the safety lockout feature is disabled, and/or before power is provided to other components of the personal vaporizer 10. For example, a first one of the sensors 18 may need to be touched twice, followed by three touches of a second one of the sensors 18, followed by two more touches of the first one of the sensors 18, before the controller controls the atomizer 14 to vaporize the liquid material contained in the cartridge 16. This is merely an example of one type of touch sequence that may be used to enable the device, and in some example embodiments such a sequence may be used to merely power the device on before a simultaneous touching is needed for the controller to control the atomizer 14 to perform the vaporizing operation.

In various example embodiments of the present general inventive concept, the sensors 18 may be provided as buttons or other types of pressure sensors that must be engaged simultaneously or in a predetermined series to overcome the safety lockout feature of the personal vaporizer 10. In other various example embodiments, the sensors 18 may be provided as any other type of touch sensors, such as heat sensors, which will sense the touch of a user. Also, while FIG. 1 illustrates the sensors 18 as being located in relatively close proximity to one another in a relatively small area of the device, different example embodiments may provide the sensors 18 as being farther away from one another, and/or on opposite sides of the device, and so on. Regardless of the placement of the sensors 18, as long as the sensors 18 are in electrical communication with the controller to indicate when the user has provided the requisite touching of the sensors, the controller may control the operation of the atomizer 14 to operate only when the authorization condition has been satisfied. As described herein, the authorization condition simply refers to a predetermined condition or procedure, other than simply switching a device to an on state with a conventional on switch, that has taken place to allow use of the personal vaporizer 10. When the personal vaporizer is in an on state, or when the authorization condition has been satisfied, the vaporization action may be actuated by the user “puffing” on the mouthpiece of the device.

Although two sensors 18 are illustrated as being provided with which to satisfy an authorization condition in FIG. 1, other example embodiments may provide three or more sensors to be engaged in a predetermined sequence and/or simultaneously to satisfy the authorization condition. Also, a user may engage a user interface, which will be described more in the example embodiments discussed herein, to change a sequence and/or number of the sensors which need to be engaged to satisfy the authorization condition. Other various example embodiments may include a personal vaporizer that is provided with only one sensor. In an example embodiment having one sensor, the sensor may be configured to be able to recognize one or more fingerprints of an authorize user in order to satisfy the authorization condition. In some example embodiments, the fingerprint reading sensor may be provided along with one or more other types of sensors used to satisfy the authorization condition. In various example embodiments, the one or more sensors must be engaged for a predetermined amount of time or any time mandated by law which engages the ability of the action of taking a pull of puff off of the personal vaporizer. In some example embodiments, there will be a predetermined amount of time that the atomizer will stay engaged based on the longest period of time it takes a person to typically smoke an actual combustible cigarette.

In various example embodiments, a sensor used to satisfy the authorization condition of the personal vaporizer 10 may be a wireless communication unit configured to sense a proximity to a paired communication device, such as a smart phone. For example, a user may wish to pair the personal vaporizer 10 to the user's smart phone such that the authorization condition of the personal vaporizer 10 is only satisfied when the personal vaporizer 10 is with a predetermined distance from the user's smart phone. The wireless communication unit provided to the personal vaporizer 10 may determine the distance from the paired smart phone by analyzing the received signal, and/or may receive distance information determined by the smart phone itself. With such an arrangement, if the user, and therefore presumably the user's smart phone, is not within a certain distance from the personal vaporizer 10, then the authorization condition is not satisfied, and the use of the personal vaporizer 10 may be prohibited. The wireless communication unit may communicate with the paired communication device through any number of communication standards, such as Wi-Fi, Bluetooth, and so on.

Various example embodiments of the present general inventive concept may include combinations of the different sensors described above, which may be utilized separately or in combination to satisfy the authorization condition. For example, a fingerprint sensor may be provided along with one or more touch sensors, and either the touch sensors or the fingerprint sensor may be utilized to satisfy the authorization condition. In other example embodiments, more than one type of sensor may need to be properly engaged in combination to satisfy the authorization condition.

FIG. 2 illustrates an exploded view of some of the components of a personal vaporizer according to another example embodiment of the present general inventive concept. As illustrated in FIG. 2, the personal vaporizer 20 shares many of the same features of the personal vaporizer 10 illustrated in FIG. 1. However, as illustrated in the block diagram of the cartridge 22 of FIG. 2, dual chambers 24,25 have been provided to the cartridge 22 to contain two different types of liquid materials which may be vaporized for inhalation by the user. In the example embodiment illustrated in FIG. 2, the cartridge 22 is provided with a microprocessor 26 that operates in conjunction with a capacitor-sensor 28 to dispense the liquid materials contained in the dual chambers 24,25. The dual chambers 24,25 allows a user to store two different substances in the personal vaporizer 20 to be vaporized and inhaled at the user's convenience. For example, a first chamber 24 may contain nicotine, and a second chamber 25 may contain a prescribed medication. The user may be able to selectively vaporize and inhale each of the substances, or may wish to have each of them vaporized to be inhaled at the same time. When operated in such a fashion, the personal vaporizer 20 allows the user to enjoy a substance such as a flavored nicotine material at the same time as inhaling an herbal substance, a medicinal drug, or other substance. According to various example embodiments of the present general inventive concept, the microprocessor 26 may be in electrical communication with the previously described controller provided to the personal vaporizer outside of the cartridge 22. In other various example embodiments, the microprocessor 26 located in the cartridge 22 may serve as the controller to control all of the operations of the personal vaporizer 20.

In the example embodiment of the present general inventive concept illustrated in FIG. 2, the microprocessor 26 is in electrical communication with the controller located in another section of the personal vaporizer 20, such as in the power source 12 section or the atomizer 14 section. The controller may recognize that a user wishes to vaporize one or both of the liquid materials contained in the dual chambers 24,25 and transmit a corresponding signal to the atomizer 14 and microprocessor 26. In various example embodiments, the microprocessor 26 in the cartridge 22 may be in electrical communication with the atomizer 14 in addition to, or instead of, the controller located outside of the cartridge 22. The microprocessor 26 controls the amount of liquid material released from each of the dual chambers 24,25 to atomizer for vaporization according to a user request, or according legal regulations, or any other desired or mandated rate. For example, a cartridge may be provided which has a controlled substance, in one or both of the dual chambers 24,25, the regulation of dosage of which is mandated by law. In such a scenario, the microprocessor 26 may be programmed to only allow a predetermined amount of one or both liquid materials to be vaporized for the user in a specific time period. For example, if only 5 grams of a liquid controlled substance in the first chamber 24 is legally allowed to be vaporized and inhaled in a four hour period, the microprocessor may prevent the substance in that first chamber 24 from being dispensed once that limit is reached, and may begin the next dosage amount after the four hour period has terminated. During the time that the liquid controlled substance is prevented from being dispensed from the first chamber 24, the liquid material contained in the second chamber 25 may continue to be dispensed, according to any corresponding preferences or regulations.

The controller and/or microprocessor 26 may also provide heating information to the atomizer 14 to adjust a heating temperature used for vaporization according to which liquid material or liquid materials are being dispensed for vaporization. For example, a liquid material contained in the first chamber 24 may need a different level of heating for optimal vaporization than does a liquid material contained in the second chamber 25. As another example, different levels of heat may be optimal depending on the amounts of liquid materials dispensed from the dual chambers 24,25 during simultaneous vaporization of both liquid materials.

The example embodiment of the personal vaporizer 20 illustrated in FIG. 2 is shown to have three main components: the power source 12, the atomizer 14, and the cartridge 22. These components may be assembled together in a host of ways, depending on different various example embodiments of the present general inventive concept. For example, the atomizer 14 may have a threaded protrusion that screws into a threaded opening in the power source. As another example, the cartridge 22 may have a male-female sliding arrangement with the atomizer 14. In some example embodiments, the connection will have electrical communication leads so that electrical components in one section of the personal vaporizer 20 may be in electrical communication with electrical components in another section. As an example, the controller which controls operations of the atomizer 14 and power source 12 may be located in the same section as the power source 12, or the atomizer 14, or even the cartridge 20. Although the sections of the personal vaporizer 20 are listed as the power source 12, atomizer 14, and cartridge 22, it will be understood by one skilled in the art that those sections of the personal vaporizer 22 may be configured to house other components along with the respective components that give them their respective labels. For example, the power source 12 section may include on/off controls, sensors 18 used to satisfy an authorization condition, a controller to control operations of the personal vaporizer, and so on. It is also understood that while the personal vaporizer 20 illustrated in FIG. 2 is shown as having three sections assembled to be together, different example embodiments may include more or fewer physical components. For example, the power source 12 and atomizer 14 may be integrated into a single housing. As another example, the power source 12 and atomizer 14 may have separate housings from a component housing the controller, sensors, or other components.

In various example embodiments of the present general inventive concept, the power source 12 and atomizer 14 are configured to be assembled and function with a plurality of different supplied cartridges. Example embodiments of the dual chamber cartridges 22 may be prescribed and acquired from a pharmacy or packaged as a retail item. For example, a cartridge 22 that is prescribed by a medical professional, and which may be purchased at a pharmacy, may include one or more medicinal substances included in the dual chambers 24,25. The microprocessor 26 or other similar controller may be pre-programmed so as to dispense a predetermined amount of the liquid materials contained therein based on time, number of puffs by a user, and so on. The microprocessor 26 may be configured so as to not be tampered with by the user, so that the proper amount of medication may be administered at the leisure of the user, and not be exceeded. Such an example microprocessor 26 may include a communication unit to communicate with the prescribing party in a wired or wireless fashion. For example, a pharmacist may be able to communicate wirelessly with the cartridge 22 through a computer or other interface to program the dosage amounts, times, etc., corresponding to the prescribed user and liquid materials contained therein. In other various example embodiments, the pharmacist may be able to maintain communication with the cartridge even in a remote location such as the user's home through a Wi-Fi, Bluetooth, etc., or even a wired connection to adjust the dosage parameters in a case in which dosage needs to be adjusted before the cartridge is spent. Various example embodiments of the dual chamber cartridge may allow the vaporization of an included liquid material at a prescribed rate during any time that the personal vaporizer is powered or authorized to vaporizer the liquid materials. The personal vaporizer may be equipped with an LED or similar light to indicate when vaporizing is occurring, and this light may also be controlled to blink or otherwise warn a user when a dosage of a substance is exceeding, or about to exceed, recommended or allowed levels.

In other various example embodiments of the present general inventive concept, the dual-chamber cartridge may be provided without a microprocessor, and the dosage of the liquid materials contained therein may be managed by the controller provided to one of the other portions of the personal vaporizer. For example, the same controller (e.g., microprocessor chip) that controls the power supply, atomizer, and so on may also be programmed to administer doses of the liquid materials in the dual chamber cartridge according to legal or otherwise mandated or recommended amounts/times. A pharmacist or otherwise prescribing party may communicate with the controller through a computer interface, either through a wired or wireless communication, to program the dosage actions of the controller in a similar fashion to that previously described with a cartridge containing a dosage controlling microprocessor in the cartridge.

It will be understood that although the example embodiment of the personal vaporizer 20 illustrated in FIG. 2 shows both the dual chamber cartridge 22 and the authorization sensors 18, different example embodiments of the personal vaporizer may include one or more sensors 18 with only a single chamber cartridge, while still other example embodiments may include a dual chamber cartridge 22 without any such authorization sensors 18. In still other example embodiments, a cartridge with three or more chambers for containing different liquid materials may be provided according to the present general inventive concept. Different example embodiments may include personal vaporizers having a number of atomizers that correspond to the number of liquid material chambers in the cartridge, while others may include personal vaporizers having a single atomizer to vaporize the plurality of liquid materials in the plurality of chambers either simultaneously or at different times. Various example embodiments of the present general inventive concept may provide one or more atomizers in the dual chamber cartridge itself, and the cartridges may be re-usable/re-fillable after the liquid containing chambers have been emptied by a user. In various example embodiments of the present general inventive concept, the multi-chamber cartridge may be a modular cartridge wherein a housing contains the two or more cartridges configured to contain different liquid materials inside the housing, the modularity of the cartridge allowing easy attachment and detachment for use with a personal vaporizer having a corresponding coupling section to which the modular multi-chamber cartridge may be coupled. Thus, a user's convenience may be increase by being able to obtain a plurality of the multi-chamber cartridges at one time to dispense medicine or other substances over controlled times for days or weeks at a time. The user's convenience may also be increased by being able to administer more than one medicinal substance at a time, by mixing a medicinal substance with a preferred nicotine and/or flavored substance at the same time, by alternating medicinal or non-medicinal substances without having to change cartridges, and so on. In various example embodiments, the controller is configured, perhaps by a pharmacist or other prescribing party, to recognize when the cartridge is changed out, so that data concerning dosage of a medicine or other substance can be modified accordingly, as can corresponding operations of the personal vaporizer. For example, if a user has ingested a vaporization of only 2 grams of a medicinal substance before the chamber containing the substance is empty, but is intended to ingest 5 grams, the controller may determine that 3 grams of the substance is still to be vaporized in the prescribed time period, and operate the personal vaporizer accordingly upon a new cartridge containing the same substances being coupled to the personal vaporizer.

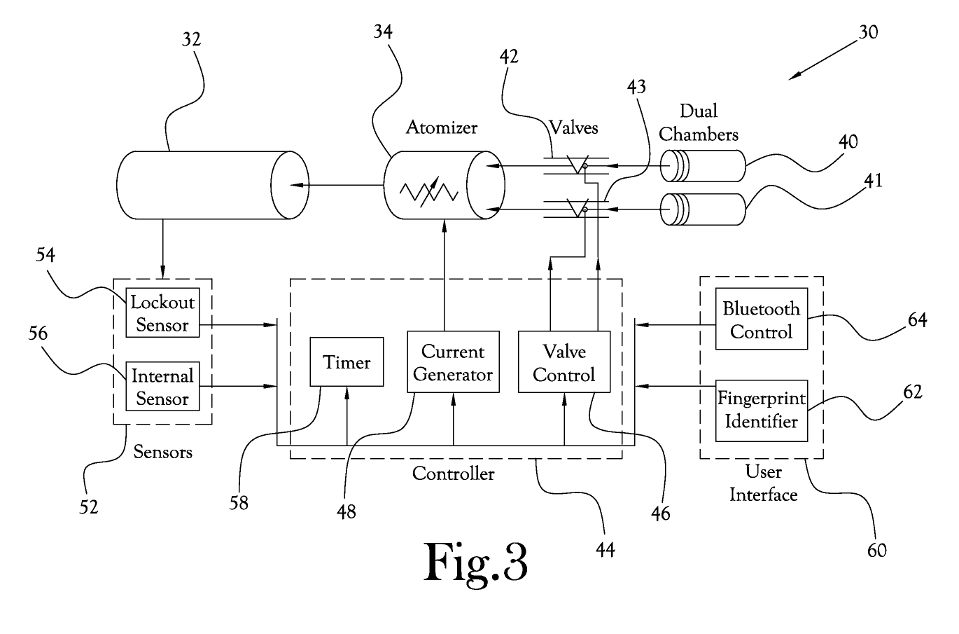

FIG. 3 illustrates a block diagram of various components of a personal vaporizer according to yet another example embodiment of the present general inventive concept. The example embodiment of the personal vaporizer 30 illustrated in FIG. 3 is provided with lockout functions as well as dual chambers 40,41 in the cartridge, but it is understood that various other example embodiment may contain only one of these features, or differently configured representations of these features. FIG. 3 illustrates, in block diagram fashion, various components of the controller 44 and other components in electrical communication with the controller 44 that are used to control various operations of the personal vaporizer 30. The controller 44 is illustrated as having a timer 58, a current generator 48, and a valve control 46 integrated with the controller 44, and the controller 44 being in electrical communication with sensors 52 and user interface 60. However, it will be understood by one skilled in the art that different combinations of these components may be included on one or several chipsets including a microprocessor, and the more or fewer components may be substituted in various example embodiments of the present general inventive concept.

As illustrated in the example embodiment of FIG. 3, the power source 32 is in electrical communication with the atomizer 34, which receives liquid materials from the dual chambers 40,41 through corresponding respective valves 42,43. The amount of liquid material allowed to pass from the dual chambers 40,41 through the valves 42,43 to the atomizer 34 may be controlled by a valve control unit 46 of the controller 44. According to any dosage regulations that may be related to the liquid materials in the dual chambers 40,41, the controller 44 may control the valve control unit 46 to open and close the valves 42,43 to allow more or less of the respective liquid materials through to the atomizer 34. For example, if a controlled substance provided in the first chamber 40 is intended to be administered at a certain flow rate and/or for a certain time, the valve control unit 46 will regulate the flow as such by operating the valve 42 to open and/or close, and prevent any further liquid from being passed from the first chamber 40 until it is proper to do so. In that same example, an uncontrolled substance may be contained in the second chamber 41, and the valve control unit 46 may allow the valve 43 to open and pass the corresponding liquid material to the user's desire. In some example embodiments, the valves 42,43 may operate simply in an open or closed state. In other various example embodiments, the valves 42,43 may open to various degrees to speed up or slow down the flow of the corresponding liquid materials. The controller 44 also controls a current generator 48 to provide a particular level of current to the atomizer in order to control a heating operation, and thus a vaporizing operation, of the atomizer 34. For example, the current generator 48 may cease providing current to the atomizer 34 to prevent further vaporization, or may increase or decrease the provided current to change the temperature used to vaporize the one or more liquid materials provided to the atomizer 34. A timer 58 may be provided such that the controller 44 controls the current generator 48 and/or valve control unit 46 to perform vaporization operations for certain times, for example, to keep up with time constraints of medicinal dosages, to allow a user to vaporize a nicotine material for a same amount of time as typically taken to smoke a combustible cigarette, to limit vaporization of nicotine or other substances to a certain amount of times in a minute, and so on.

The example embodiment of the present general inventive concept illustrated in FIG. 3 includes different types of sensors 52 configured to be used in different ways to operate the personal vaporizer 30. The lockout sensor 54 may operate as the previously described authorization sensors to indicate whether an authorization condition has been satisfied for the personal vaporizer 30 to operate. For example, the lockout sensor 54 may be a proximity sensor to detect whether the personal vaporizer 30 is within a predetermined distance from a paired communication device, a touch sensor to detect whether a predetermined number of simultaneous touches or sequence of touches has been performed by a user, a fingerprint sensor to detect whether the fingerprint is that of an authorized user, and so on. When the controller 44 determines through communication from the lockout sensors 54 that the authorization condition has been satisfied, the controller may control the current generator 48 and valve control 46 to allow vaporization operations. An internal sensor 56 may be provided to detect a drawing of air on the personal vaporizer 30 by the user, thereupon which the controller may control the atomizer and valves to perform vaporization operations as long as the authorization condition is satisfied, or for a certain amount of time after the authorization condition has been satisfied. In other words, in various example embodiments of the present general inventive concept, detection of a user's desired use by the internal sensors 56 may cause the controller 44 to perform vaporization operations if the authorization conditions are not indicated as satisfied by the lockout sensors 54.

The example embodiment illustrated in FIG. 3 also shows a user interface 60 which may be used in conjunction with the personal vaporizer 30. A fingerprint identifier 62 may be used confirm that a user is authorized to communicate with the personal vaporizer 30 through the user interface 60, and a Bluetooth control unit 64 may be used to configure various operations of the personal vaporizer 30 by communicating with the controller 44 having a communication unit (not shown). It is understood that the present general inventive concept is not limited to a Bluetooth control unit, and that the user interface may also communicate by wired or other wireless means of communication. The user interface 60 may be used to communicate with the personal vaporizer 30 through a remote server-based application, a remote cloud-based application, a mobile communication device-based application, and so on. In various example embodiments, the user interface 60 may be accessed to notify the controller as to dosage amounts and times that correspond to the liquid materials contained in the dual chambers 40,41. In other various example embodiments, the user interface 60 may be used to change the authorization conditions used by the controller to allow use of the personal vaporizer 30, the authorization conditions and associated data being stored in a memory (not shown) in electrical communication with the controller 44. The controller 44 may include software used to control the various components of the personal vaporizer 30, and the software may be computer readable instructions stored in the memory.

For example, in the example embodiment illustrated in FIG. 3, the lockout sensors 54 may be two lockout pressure sensors. When the two lockout pressure sensors continuously detect pressure at the same time for 5 seconds (or any arbitrary amount of time) the personal vaporizer 30 may be turned on, and the controller 44 may go to a standby mode. At the same time, a 4-minute (or any arbitrary amount of time) timer may be activated, in the timer 58, which determines the time after which the personal vaporizer automatically turns off. In various example embodiments, the lockout feature of the device can also be implemented using the fingerprint identifier 62, either in combination with, or in place of, the pressure sensors. When a user inhales through the mouthpiece, the internal sensors 56 may detect the breathing and the valve control unit 46 may be activated by the controller 44. Although the timer 58, current generator 48, and valve control unit 46 may be referred to herein as being controlled by the controller 44, it is also possible to consider these units as being part of the controller 44 itself. Alternatively, these units may be provided separately from the controller 44. Depending upon the configuration (which may be controlled according to programming by the user interface), either one or both valves 42,43 may be opened. The controller 44 may control the valve control signal to be pulsed, and the width of the pulse may be configurable so that the duration or the amount of the medicine/nicotine (or other material) dispensed can be controlled. Meanwhile, the atomizer 34 may also be activated by the controller 44 supplying current from the current generator 48. The amount of current supplied may be controlled so as to enable the atomizer 34 to condition the heating according to specific requirements corresponding to the liquid materials in the dual chambers 40,41. When the user stops inhaling, and thus the internal sensors 56 do not detect the breath of the user, the valve control unit 46 and the current generator 48 signals may be deactivated until the next inhalation. In the example embodiment illustrated in FIG. 3, the valves 42,43 may be provided to a modular cartridge including the dual chambers 40,41. In such an example embodiment, the atomizer 34 may include a single burning element to vaporize the liquid materials received from the dual chambers 40,41, or may include a plurality of atomizers corresponding to a number of valves and dual chambers in the cartridge. Thus, such a modular cartridge is able to be changed out on the personal vaporizer with little trouble by the user. In other example embodiments, the modular cartridge may include a plurality of chambers configured to contain liquid materials, and the corresponding valves may be provided as part of the personal vaporizer body outside of the cartridge, such as in the atomizer 34 section of the personal vaporizer.

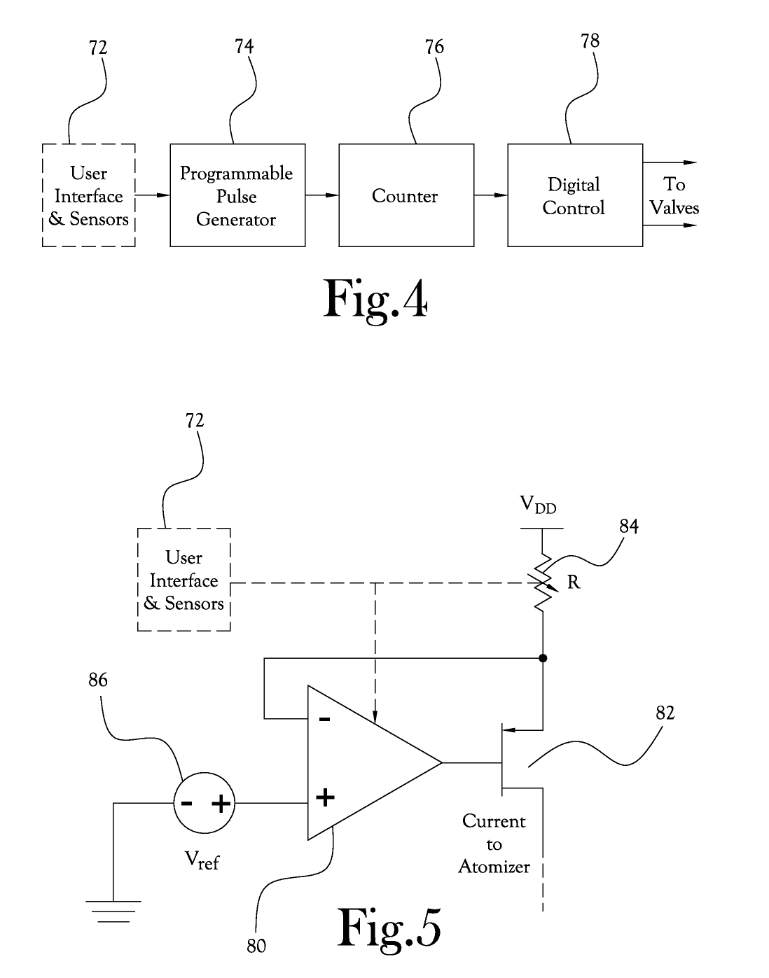

FIG. 4 illustrates a block diagram of various components of the valve control unit 46 of FIG. 3 according to an example embodiment of the present general inventive concept. As seen in the example embodiment illustrated in FIG. 4, the valve control unit 46 of the controller 44 may include a programmable pulse generator 74 to receive signals from the user interface and sensors (indicated together as a single block in FIG. 4 with the identifier 74 for simplicity) and generate a pulse signal corresponding to the received signals, a counter 76 configured to count a number of pulses generated by the programmable pulse generator 74, and a digital control 78 to control the valves 42,43 according to the received signals. In various example embodiments, the programmable pulse generator 74 may be capable of producing pulses with a period within the range of 1 μs to 1 ms, which may be controlled, for example, by changing the values of variable resistors within the circuit. Such variable resistors may be implemented, for example, using arrays of MOS-bipolar pairs, and the values of the variable resistors may be changed by turning on/off different branches of the arrays. The generated pulse may be used as the clock for a 10-bit counter, which may be optimal in some example embodiments because designing an integrated pulse generator with a period in the 1 s range may be impractical. In various example embodiments, the Most Significant Bit (MSB) of the counter 76 may be used as one of the inputs of the digital control block 78 which may include simple digital gates. The configuration set by the user interface 72 and the MSB signal may separately control the valves of the two chambers so that different dosage amounts, rates, etc., may be applied to the different liquid materials contained in the dual chambers 40,41.

FIG. 5 illustrates a schematic diagram of the current generator 48 of FIG. 3 according to an example embodiment of the present general inventive concept. In the example embodiment illustrated in FIG. 5, the current generator includes a variable resistor 84 and amplifier 80 receiving signals from the user interface and sensors 72, and a p-channel MOSFET 82 is controlled accordingly by the output of the variable resistor 84 and amplifier 80 to adjust an amount of current supplied to the atomizer 34. According to various example embodiments of the present general inventive concept, the variable resistor 84 may be implemented using MOS-bipolar pairs and by changing the value of the resistor, the current going into the atomizer 34 may be controlled. Therefore, different levels of heat may be applied to vaporize different liquid materials, or combinations of different liquid material. The current supplied may depend on several factors such as, for example, the type of liquid material, the amount of liquid material passed by the valves 42,43, whether a predetermined time for vaporizing the liquid materials has elapsed, and so on.

According to various example embodiments, a user interface operating through, for example, a Bluetooth control, may enable the user or the pharmacist to set the different parameters within the device such as the duration of medicine/nicotine dispense on each puff, controlling the valves of the two chambers, and the amount of heating in the atomizer 34. Through an interfacing software and electrical communication connectivity, the configurations can be determined according to specific requirements, whether according to preference, legal mandate, material parameter, and/or other concerns.

Various example embodiments of the present general inventive concept may provide a personal vaporizer including a cartridge configured to contain a liquid material, an atomizer configured to vaporize the liquid material so as to be inhaled by a user, a controller configured to control the atomizer, and one or more sensors configured to be in electrical communication with the controller, wherein the controller is configured to control the atomizer to vaporize the liquid material only when an authorization condition is satisfied, the authorization condition being determined according to signals received from the one or more sensors. The one or more sensors may include two sensors provided on the personal vaporizer a predetermined distance from one another, and the controller may be configured to determine the authorization condition as being satisfied only when both of the two sensors are touched simultaneously for a predetermined amount of time. The two sensors may be pressure sensors. The two sensors may be heat sensors. The one or more sensors may include a fingerprint sensor provided on the personal vaporizer and configured to recognize one or more fingerprints of one or more users authorized to use the personal vaporizer, and the controller may be configured to determine the authorization condition as being satisfied only when a fingerprint of an authorized user is detected for a predetermined amount of time. The one or more sensors may include a proximity sensor provided to the personal vaporizer and configured to wirelessly communicate with a remote communication device to determine a distance from the personal vaporizer to the remote communication device, and the controller may be configured to determine the authorization condition as being satisfied only when the personal vaporizer is within a predetermined distance from the remote communication device. The remote communication device may be a mobile communication device that has been previously paired with the personal vaporizer. The personal vaporizer may further include a power source configured to provide power to the controller and atomizer. The power may be provided to the one or more sensors from the power source or the controller. The personal vaporizer may further include a power switch configured to switch the personal vaporizer between an on and off state, and the power may not be provided to the controller and atomizer when the personal vaporizer is in the off state.

Various example embodiments of the present general inventive concept may provide a personal vaporizer including a cartridge configured to accommodate a plurality of chambers each configured to contain a liquid material, an atomizer configured to vaporize one or more liquid materials received from one or more of the chambers, a plurality of valves respectively provided to the plurality of chambers and configured to selectively control flow of the one or more liquid materials to the atomizer, and a controller configured to control the valves to allow the flow of the one or more liquid materials, and to control the atomizer to vaporize the one or more liquid materials received from the one or more of the chambers. The controller may be configured to control an amount of flow through each respective valve according to a particular liquid material regulated by the respective valve. The cartridge may be configured to accommodate a first chamber containing a first liquid material, and a second chamber containing a second liquid material, a first valve is configured to be coupled to the first chamber, and a second valve is configured to be coupled to the second chamber, and the controller controls the first valve to release an amount of the first liquid material to the atomizer, and the second valve to release an amount of the second liquid material to the atomizer, the amount of the first liquid material being different than the amount of the second liquid material. The plurality of valves may be accommodated inside the cartridge, the cartridge being configured to be readily attached and detached from the personal vaporizer. The personal vaporizer may further include a wireless communication unit configured to wirelessly communicate with a communication device to receive dosage instructions corresponding to at least one of the one or more liquid materials. The personal vaporizer may further include a memory in which to store the dosage instructions corresponding to at least one of the one or more liquid materials. The controller may be configured to control the valves and atomizer according to the stored dosage instructions. The personal vaporizer may further include a cartridge sensor to detect when a new cartridge has been attached to the personal vaporizer. The controller may be configured to control the communication unit to obtain updated dosage instructions corresponding to the new cartridge being attached to the personal vaporizer. The personal vaporizer may further include a microprocessor provided in the cartridge and configured to communicate and store dosage information corresponding to the liquid material in the respective chambers of the cartridge. The microprocessor may be configured to control an amount of flow through each respective valve according to the dosage information and control signals from the controller.

Various example embodiments of the present general inventive concept may provide a cartridge to supply a liquid material to a personal vaporizer, the cartridge including a plurality of chambers each configured to contain a liquid material, a housing to at least partially enclose the plurality of chambers, and a mating portion to couple the cartridge to the personal vaporizer such that the cartridge is readily attachable and detachable to the personal vaporizer as a modular component. The cartridge may further comprise a plurality of valves respectively provided to the plurality of chambers and configured to selectively control flow of the liquid material contained in each of the chambers. The cartridge may further include a microprocessor configured to be in electrical communication with the plurality of valves to control respective operations of the valves. The cartridge may further include a memory configured to store dosage instructions corresponding to the liquid material in the plurality of chambers, wherein the microprocessor controls the operations of the valves according to the dosage instructions. Each of the chambers may contain a different liquid material containing, for example, nicotine, prescription drugs, flavorings, and so on.

Various example embodiments of the present general inventive concept may provide a personal vaporizer including a cartridge configured to accommodate a plurality of chambers each configured to contain a liquid material, an atomizer configured to vaporize the liquid material so as to be inhaled by a user, a controller configured to control the atomizer, and one or more sensors configured to be in electrical communication with the controller, wherein the controller is configured to control the atomizer to vaporize the liquid material only when an authorization condition is satisfied, the authorization condition being determined according to signals received from the one or more sensors.

According to various example embodiments of the present general inventive concept, a personal vaporizer may be provided with one or more lockout sensors that allow the personal vaporizer to operate, and thus vaporize a liquid material, only when a predetermined authorization condition is satisfied. According to other various example embodiments, a personal vaporizer may be provided with a cartridge having two or more chambers in which different liquid materials may be respectively stored. According to still other various example embodiments, a modular multi-chamber cartridge in which two or more different liquid materials may be respectively stored is provided which mates with a separately provided personal vaporizer such that different liquid materials may be vaporized, either together or separately, without a user having to change liquid material cartridges connected to the personal vaporizer.

As a non-exhaustive illustration only, a communication terminal/device/unit described herein may refer to mobile devices such as a cellular phone, a personal digital assistant (PDA), a digital camera, a portable game console, and an MP3 player, a portable/personal multimedia player (PMP), a handheld e-book, a portable lab-top PC, a global positioning system (GPS) navigation, and devices such as a desktop PC, a high definition television (HDTV), an optical disc player, a setup box, and the like capable of wireless communication or network communication consistent with that disclosed herein.

Numerous variations, modifications, and additional embodiments are possible, and accordingly, all such variations, modifications, and embodiments are to be regarded as being within the spirit and scope of the present general inventive concept. For example, regardless of the content of any portion of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated.

It is noted that the simplified diagrams and drawings included in the present application do not illustrate all the various connections and assemblies of the various components, however, those skilled in the art will understand how to implement such connections and assemblies, based on the illustrated components, figures, and descriptions provided herein, using sound engineering judgment. Numerous variations, modification, and additional embodiments are possible, and, accordingly, all such variations, modifications, and embodiments are to be regarded as being within the spirit and scope of the present general inventive concept.

While the present general inventive concept has been illustrated by description of several example embodiments, and while the illustrative embodiments have been described in detail, it is not the intention of the applicant to restrict or in any way limit the scope of the general inventive concept to such descriptions and illustrations. Instead, the descriptions, drawings, and claims herein are to be regarded as illustrative in nature, and not as restrictive, and additional embodiments will readily appear to those skilled in the art upon reading the above description and drawings. Additional modifications will readily appear to those skilled in the art. Accordingly, departures may be made from such details without departing from the spirit or scope of applicant's general inventive concept.

1. A personal vaporizer comprising:
a cartridge configured to contain a liquid material;
an atomizer configured to vaporize the liquid material so as to be inhaled by a user;
a controller configured to control the atomizer; and
one or more sensors configured to be in electrical communication with the controller;
wherein the controller is configured to control the atomizer to vaporize the liquid material only when an authorization condition is satisfied, the authorization condition being determined according to signals received from the one or more sensors.

2. The personal vaporizer of claim 1, wherein the one or more sensors comprises two sensors provided on the personal vaporizer a predetermined distance from one another; and
and wherein the controller is configured to determine the authorization condition as being satisfied only when both of the two sensors are touched simultaneously for a predetermined amount of time.

3. The personal vaporizer of claim 2, wherein the two sensors are pressure sensors.

4. The personal vaporizer of claim 2, wherein the two sensors are heat sensors.

5. The personal vaporizer of claim 1, wherein the one or more sensors comprises a fingerprint sensor provided on the personal vaporizer and configured to recognize one or more fingerprints of one or more users authorized to use the personal vaporizer; and
wherein the controller is configured to determine the authorization condition as being satisfied only when a fingerprint of an authorized user is detected for a predetermined amount of time.

6. The personal vaporizer of claim 1, wherein the one or more sensors comprises a proximity sensor provided to the personal vaporizer and configured to wirelessly communicate with a remote communication device to determine a distance from the personal vaporizer to the remote communication device; and
wherein the controller is configured to determine the authorization condition as being satisfied only when the personal vaporizer is within a predetermined distance from the remote communication device.

7. The personal vaporizer of claim 6, wherein the remote communication device is a mobile communication device that has been previously paired with the personal vaporizer.

8. The personal vaporizer of claim 1, further comprising a power source configured to provide power to the controller and atomizer.

9. The personal vaporizer of claim 8, wherein power is provided to the one or more sensors from the power source or the controller.

10. The personal vaporizer of claim 8, further comprising a power switch configured to switch the personal vaporizer between an on and off state, wherein power is not provided to the controller and atomizer when the personal vaporizer is in the off state.

11. A personal vaporizer comprising:
a cartridge configured to accommodate a plurality of chambers each configured to contain a liquid material;
an atomizer configured to vaporize one or more liquid materials received from one or more of the chambers;
a plurality of valves respectively provided to the plurality of chambers and configured to selectively control flow of the one or more liquid materials to the atomizer; and
a controller configured to control the valves to allow the flow of the one or more liquid materials, and to control the atomizer to vaporize the one or more liquid materials received from the one or more of the chambers.

12. The personal vaporizer of claim 11, wherein the controller is configured to control an amount of flow through each respective valve according to a particular liquid material regulated by the respective valve.

13. The personal vaporizer of claim 12, wherein the cartridge is configured to accommodate a first chamber containing a first liquid material, and a second chamber containing a second liquid material;
a first valve is configured to be coupled to the first chamber, and a second valve is configured to be coupled to the second chamber; and
the controller controls the first valve to release an amount of the first liquid material to the atomizer, and the second valve to release an amount of the second liquid material to the atomizer, the amount of the first liquid material being different than the amount of the second liquid material.

14. The personal vaporizer of claim 11, wherein the plurality of valves are accommodated inside the cartridge, the cartridge being configured to be readily attached and detached from the personal vaporizer.

15. The personal vaporizer of claim 11, further comprising a wireless communication unit configured to wirelessly communicate with a communication device to receive dosage instructions corresponding to at least one of the one or more liquid materials.

16. The personal vaporizer of claim 15, further comprising a memory in which to store the dosage instructions corresponding to at least one of the one or more liquid materials.

17. The personal vaporizer of claim 16, wherein the controller is configured to control the valves and atomizer according to the stored dosage instructions.

18. The personal vaporizer of claim 17, further comprising a cartridge sensor to detect when a new cartridge has been attached to the personal vaporizer.

19. The personal vaporizer of claim 18, wherein the controller is configured to control the communication unit to obtain updated dosage instructions corresponding to the new cartridge being attached to the personal vaporizer.

20. The personal vaporizer of claim 11, further comprising a microprocessor provided in the cartridge and configured to communicate and store dosage information corresponding to the liquid material in the respective chambers of the cartridge.

21. The personal vaporizer of claim 20, wherein the microprocessor is configured to control an amount of flow through each respective valve according to the dosage information and control signals from the controller.

22. A cartridge to supply a liquid material to a personal vaporizer, the cartridge comprising:
a plurality of chambers each configured to contain a liquid material;
a housing to at least partially enclose the plurality of chambers; and
a mating portion to couple the cartridge to the personal vaporizer such that the cartridge is readily attachable and detachable to the personal vaporizer as a modular component.

23. The cartridge of claim 22, further comprising a plurality of valves respectively provided to the plurality of chambers and configured to selectively control flow of the liquid material contained in each of the chambers.

24. The cartridge of claim 23, further comprising a microprocessor configured to be in electrical communication with the plurality of valves to control respective operations of the valves.

25. The cartridge of claim 24, further comprising a memory configured to store dosage instructions corresponding to the liquid material in the plurality of chambers, wherein the microprocessor controls the operations of the valves according to the dosage instructions.
